(19) United States
(12) Patent Application Publication
(10) Pub. No.: US 2017/0022142 A1
(43) Pub. Date: Jan. 26, 2017

(54) METHOD FOR THE PRODUCTION OF HIGH-PURITY GLYCEROL DIMETHACRYLATE

(71) Applicants: Joachim KNEBEL, Alsbach-Hähnlein (DE); Volker HERZOG, Darmstadt (DE); Maik CASPARI, Alsbach-Haehnlein (DE); EVONIK RÖHM GMBH, Darmstadt (DE)

(72) Inventors: Joachim KNEBEL, Alsbach-Haehnlein (DE); Volker HERZOG, Darmstadt (DE); Maik CASPARI, Alsbach-Haehnlein (DE)

(73) Assignee: Evonik Roehm GmbH, Darmstadt (DE)

(21) Appl. No.: 15/119,327

(22) PCT Filed: Feb. 10, 2015
(86) PCT No.: PCT/EP2015/052678

§ 371 Date: Aug. 16, 2016

(30) Foreign Application Priority Data
Feb. 18, 2014 (DE) 10 2014 202 902.3

(51) Int. Cl. C07C 67/56

(52) U.S. Cl. CPC C07C 67/56

(57) ABSTRACT

The present invention claims a process for preparing high-purity glycerol dimethacrylate comprising <500 ppm of glycidyl methacrylate, characterized in that an aftertreatment with acidic adsorbent is effected.

The present invention describes a process for preparing high-purity glycerol dimethacrylate having a residual glycidyl methacrylate content of <500 ppm by aftertreatment with an acidic adsorbent.

The prior art describes processes for preparing glycerol di(meth)acrylate from (meth)acrylic acid and glycidyl (meth)acrylate in the presence of catalysts. It is preferable to use quaternary ammonium salts as catalysts.

A few per cent of unconverted glycidyl methacrylate remain in the product mixture in this reaction. It is known that glycidyl methacrylate exhibits genotoxicity in in vitro tests (OECD SIDS report “Glycidyl methacrylate”, 2000).

EP 0054700 describes a process for glycerol dimethacrylate. Here, glycidyl methacrylate is added to a mixture of methacrylic acid, benzyltriethylammonium chloride and p-methoxyphenol at a temperature of 80° C. After work-up with sodium carbonate solution and drying, the mixture is subjected to a fractional distillation. The yield is only 75%. In addition, the product polymerizes in the distillative purification.

It was an object of the present invention to provide a process for preparing glycerol dimethacrylate, wherein a high-purity glycerol dimethacrylate comprising only small amounts of glycidyl methacrylate and small amounts of mono- and triester is produced.

The object was achieved by a process for preparing high-purity glycerol dimethacrylate, characterized in that glycerol dimethacrylate comprising glycidyl methacrylate is aftertreated with an acidic adsorbent.

It was found that, surprisingly, there is no disproportionation of the diester to form mono- and triesters. The person skilled in the art would expect that disproportionation would be effected when an acidic adsorbent is used. However, it was found that there is no disproportionation to form mono- and triesters and, in addition, unconverted glycidyl methacrylate is removed very selectively when use is made of acidic ion exchangers, for example DOWEX M31 (DOW, USA) or Amberlyst 15 (DOW, USA).

It was found that the aftertreatment with the acidic adsorbent can be effected at low temperatures. Consequently, polymerization of the product on work-up is prevented.

The glycidyl methacrylate content can be lowered to below 500 ppm in the product mixture with the process according to the invention. The aftertreated glycerol dimethacrylate preferably comprises <300 ppm, more preferably <100 ppm, of glycidyl methacrylate.

Using the acidic adsorbent does not lead to disproportionation of the glycerol dimethacrylate. Surprisingly, a residual content of <2000 ppm of mono- and triester is achieved. Depending on the reaction conditions, reaction mixtures were prepared comprising <1000 ppm, in some cases even less than 500 ppm, of mono- and triester.

To prevent polymerization, the reaction is effected at a temperature between 0 and 80° C., preferably at a temperature between 5 and 50° C., more preferably at room temperature.

The adsorbent is stirred into the reaction solution and subsequently separated by filtration. Alternatively, the reaction solution can also be passed through a column packed with adsorbent.

A continuous process regime is described in Example 3. Here, the glycerol dimethacrylate comprising glycidyl methacrylate and the acidic adsorbent Dowex M31 are used.

The high-purity glycerol dimethacrylate is used in polymers for coating metal surfaces.

### EXAMPLES

### Example 1

300 g of glycerol dimethacrylate comprising 88.8% by weight of glycerol dimethacrylate and 0.63% by weight of glycidyl methacrylate is admixed with 30 g of DOWEX M31 from DOW, USA (activated and dried) in a 500 ml Erlenmeyer flask and stirred at 25 CC for 2 h with a magnetic stirrer. The suspension is subsequently filtered through a rapid filter sieve (240 pm). GC analysis gives 89.4% by weight of glycerol dimethacrylate and 0.039% by weight of glycidyl methacrylate following the treatment with adsorbent.

### Example 2

A glass column (50 cm in height, 3 cm in diameter) with a glass frit is packed with 100 g of Dowex M31. 1000 g of glycerol dimethacrylate comprising 88.8% by weight of glycerol dimethacrylate and 0.63% by weight of glycidyl methacrylate is then continuously passed through the ion exchanger fixed bed at a feed rate of 1 ml/min via a 500 ml metering funnel. GC analysis gives 89.9% by weight of glycerol dimethacrylate and 0.002% by weight of glycidyl methacrylate following the treatment with adsorbent.

### Example 3

Continuous Purification

As per Example 2, with the exception that a total of 3000 g of glycerol dimethacrylate comprising 88.8% by weight of glycerol dimethacrylate and 0.63% by weight of glycidyl methacrylate is pumped through the glass column and nine succesive samples of 241 g, 988 g, 222 g, 223 g, 194 g, 256 g, 248 g, 363 g and 167 g are taken. The proportion of glycidyl methacrylate present is 0,012% by weight, 0.026% by weight, 0.047% by weight, 0.040% by weight, 0.031% by weight, 0.029% by weight, 0.044% by weight, 0.050% by weight and 0.049% by weight.

### Comparative Example 1

500 g of glycerol dimethacrylate comprising 88.8% by weight of glycerol dimethacrylate and 0.63% by weight of glycidyl methacrylate is admixed with 25 g of Tonsil L80 FF (acid-activated Montmorillonite from Sudchemie) in a 1000 ml Erlenmeyer flask and stirred at 25° C. for 3 h with a magnetic stirrer. The suspension is subsequently filtered through a pressure filter with a Seitz K 800 filtering layer. GC analysis gives 88.6% by weight of glycerol dimethacrylate and 0.59% by weight of glycidyl methacrylate following the treatment with adsorbent.

1. Process for preparing high-purity glycerol dimethacrylate, characterized in that glycerol dimethacrylate comprising glycidyl methacrylate is aftertreated with an acidic adsorbent.

2. Process according to claim 1, characterized in that the aftertreated glycerol dimethacrylate comprises <500 ppm of glycidyl methacrylate.

3. Process according to claim 1, characterized in that the aftertreated glycerol dimethacrylate comprises <300 ppm of glycidyl methacrylate.

4. Process according to claim 1, characterized in that the aftertreated glycerol dimethacrylate comprises <100 ppm of glycidyl methacrylate.

5. Process according to any one of the preceding claims, characterized in that the aftertreated glycerol dimethacrylate comprises <2000 ppm of mono- or triester.

6. Process according to any one of the preceding claims, characterized in that the aftertreated glycerol dimethacrylate comprises <1000 ppm of mono- or triester.

7. Process according to any one of the preceding claims, characterized in that the aftertreated glycerol dimethacrylate comprises <500 ppm of mono- or triester.

8. Process according to any one of the preceding claims, characterized in that the acidic adsorbent is a cation exchanger.

9. Process according to any one of the preceding claims, characterized in that the aftertreatment is carried out in a fixed bed.

10. Process according to any one of the preceding claims, characterized in that the aftertreatment is carried out via a column.

11. Process according to any one of the preceding claims, characterized in that the aftertreatment is carried out at a temperature between 5° C. and 50° C.
